(19) United States
(12) Patent Application Publication
(10) Pub. No.: US 2025/0223184 A1
(43) Pub. Date: Jul. 10, 2025

(54) COMPOSITE OF HYDROTALCITE COMPOUND AND AMINO ACID

(71) Applicant: SETOLAS Holdings, Inc., Takamatsu-shi, Kagawa (JP)

(72) Inventors: Hiroyuki SUGIYAMA, Sakaide-shi (JP); Atsushi OKADA, Sakaide-shi (JP)

(73) Assignee: SETOLAS Holdings, Inc., Takamatsu-shi, Kagawa (JP)

(21) Appl. No.: 18/853,335

(22) PCT Filed: Mar. 30, 2023
(86) PCT No.: PCT/JP2023/013328

§ 371 Date: Oct. 1, 2024

(30) Foreign Application Priority Data
Apr. 13, 2022 (JP) 2022-066617
Nov. 11, 2022 (JP) 2022-181203

(51) Int. Cl. C01F 7/785; C09D 7/40; C09D 7/61; C09D 129/04

(52) U.S. Cl. CPC C01F 7/785; C09D 7/61; C09D 7/67; C09D 129/04; C01P 2004/64

(57) ABSTRACT

The present invention provides a composite of a hydrotalcite compound and an amino acid in which the aspect ratio is high and it is possible to control colorability. The composite includes a hydrotalcite compound and an amino acid. The composite has an aspect ratio of 85 or more. The Y.I. value of the composite is 0-5. The Y.I. value indicates yellowness. The content of the amino acid in the composite is more than 0 mass % and equal to or less than 10.0 mass %. The content of the amino acid is the content with respect to the total mass of the composite.

### FIELD

The present invention relates to a complex of a hydrotalcite compound and an amino acid.

### BACKGROUND

Hydrotalcite compounds, including hydrotalcite and calcinated products thereof, exhibit ion exchange capacity and are used for a variety of purposes, such as being combined with resins to reduce resin degradation. Such hydrotalcite compounds are used in known methods that utilize amino acids to further increase the aspect ratio (PTL 1 and NPL 1). With the methods disclosed in PTL 1 and NPL 1 it is possible to obtain complexes of hydrotalcite compounds and amino acids.

### CITATION LIST

### Patent Literature

[PTL 1] International Patent Publication No. WO2019/092453

### Non Patent Literature

[NPL 1] Nature Communications (2019) 10:2398, High gas barrier coating using non-toxic nanosheet dispersions for flexible food packaging film.

### SUMMARY

### Technical Problem

However, the methods disclosed in PTL 1 and NPL 1 have been associated with the problem of coloration when the aspect ratio of the hydrotalcite compound becomes too high, thus limiting the range of use of the complex.

It is therefore an object of the present invention to provide a complex with a wider range of utility.

### Solution to Problem

As a result of much diligent research with the goal of achieving the aforementioned object, the present inventors found that a complex with a wider range of utility can be obtained by a complex of a hydrotalcite compound and an amino acid that allows colorability to be controlled while providing a high aspect ratio.

The present invention has been completed based on this finding and includes the following aspects.

### (First Disclosure)

According to the first disclosure, the complex comprises a hydrotalcite compound and an amino acid. The complex has an aspect ratio of 85 or higher. The complex has a Y.I. value of 0 to 5. Y.I. is a value representing the yellowness index. The complex has an amino acid content of greater than 0 mass % and 10.0 mass % or lower. The amino acid content is the content with respect to the total mass of the complex.

### (Second Disclosure)

The second disclosure is the first disclosure wherein the amino acids are glycine compounds.

### (Third Disclosure)

The third disclosure is the first disclosure or second disclosure wherein the hydrotalcite compound is represented by the following formula (1).

(M²⁺)_{1-X}(M³⁺)_{X}(OH)₂(Aⁿ⁻)_{X/n}·mH₂O (1)

(In formula (1), M²⁺ represents a divalent metal cation. M³⁺ represents a trivalent metal cation. Aⁿ⁻ represents an n-valent anion. X is a number satisfying the inequality 0.17<X<0.36. In formula (1), n is a number from 1 to 6. In formula (1), m is a number satisfying the inequality 0<m<1.80.)

### (Fourth Disclosure)

The fourth disclosure is any of the first disclosure to third disclosure wherein the thickness of the primary particles of the complex is 20 nm or smaller. The complex has an aspect ratio of 100 or higher.

### (Fifth Disclosure)

The fifth disclosure is a coating solution. The coating solution comprises a complex as described for any of the first disclosure to fourth disclosure. The coating solution comprises a polymer.

### (Sixth Disclosure)

The sixth disclosure is the fifth disclosure wherein the polymer is a water-soluble polymer.

### (Seventh Disclosure)

The seventh disclosure is the sixth disclosure wherein the water-soluble polymer is polyvinyl alcohol.

### (Eighth Disclosure)

The eighth disclosure is a film. The film has a covering layer.

The covering layer is formed by a coating solution as described for any of the fifth disclosure to seventh disclosure.

### (Ninth Disclosure)

The ninth disclosure is the eighth disclosure wherein the thickness of the covering layer is 1 μm to 1000 μm.

### Advantageous Effects of Invention

According to the invention it is possible to provide a complex of a hydrotalcite compound and an amino acid which has a high aspect ratio and allows colorability to be controlled.

### BRIEF DESCRIPTION OF DRAWINGS

FIG. 1 is a schematic view of a film production apparatus that produces a packaging film according to an embodiment of the invention.

FIG. 2 is a set of photographs showing slurry solutions after the heating step for Example 1 to Example 3 of the invention and Comparative Example 1 to Comparative Example 3, and photographs of slurry solutions after heating to 100° C. in the heating step, for Comparative Example 4 to Comparative Example 6.

### DESCRIPTION OF EMBODIMENTS

The complex of a hydrotalcite compound and an amino acid according to the invention (hereunder also referred to as “complex of the invention”) will now be described in detail by preferred embodiments. The phrase “complex of a hydrotalcite compound and an amino acid”, for the purpose of the present specification, means that the amino acid(s) chemically modify the hydrotalcite compound.

### [Complex]

The complex according to one embodiment of the invention is a complex of a hydrotalcite compound and an amino acid having an aspect ratio of 85 or higher. The complex of the embodiment has a Y.I. value of 0 to 5, as the value representing the yellowness index of the complex. The complex of the embodiment has an amino acid content of greater than 0 mass % and 10.0 mass % or lower, with respect to the total mass of the complex.

The complex of the embodiment, having a high aspect ratio, has multiple complex molecules arranged in parallel in the in-plane direction of the covering layer within the covering layer, after the covering layer has been formed, thus allowing an excellent gas barrier property to be exhibited. The complex of the embodiment has a Y.I. value within a specified range of 5 or lower, while the amino acid content is also within a specified range of 10.0 mass % or lower. The complex of the embodiment can therefore form a highly transparent covering layer without coloration such as yellowish-brown coloration. Without being constrained by theory, it is thought that the action mechanism by which a highly transparent covering layer is obtained may be described as follows.

First, the complex of the embodiment can be obtained by a production method comprising a solution preparation step, in which a slurry solution comprising a mixture of a complex metal oxide obtained by calcinating a hydrotalcite compound precursor, and an amino acid release agent (such as glycine), are mixed, and a heating step in which the slurry solution prepared in the solution preparation step is heated. This production method is advantageous in that using an amino acid in the release agent produces interlayer separation of the hydrotalcite compound, thereby producing a hydrotalcite compound having a small thickness with primary particles. However, in the heating step in which the hydrotalcite compound is detached, peptide bonding between the amino acids is accelerated under alkaline conditions, forming polyamino acids (for example, peptides such as polyglycine). Peptides such as polyglycine produce yellowing with extended chains, causing yellowness to increase as the molecular chains lengthen. When a complex of a hydrotalcite compound and amino acids that include such long-chain peptides (polypeptides) is combined with a polymer in a coating solution, the transparency and aesthetic quality of the resulting covering layer is impaired.

For the embodiment, limiting the slurry concentration and amino acid concentration to within specified ranges in the solution preparation step can help prevent formation of peptide bonds between the amino acids even under alkaline conditions, so that production of polyamino acids (peptides) can be inhibited. As a result, it is possible to reduce yellowing in the obtained complex. It is thus possible to obtain a complex with a high aspect ratio of 85 or higher, while having a Y.I. value (yellowness index) of 0 to 5 and an amino acid content of greater than 0 mass % and 10.0 mass % or lower. Such a complex may be mixed into a coating solution together with a polymer to form a covering layer exhibiting both a high gas barrier property and high transparency. The complex of the embodiment can thus be used for a wide variety of purposes that require gas barrier properties, without sacrificing transparency or aesthetic quality. For example, when a coating solution containing the complex of the embodiment is applied onto a film, it can impart a high gas barrier property to the film without impairing the transparency.

In the solution preparation step described above for the complex of the embodiment, the complex metal oxide obtained by calcinating the hydrotalcite compound precursor may be hydrated using an amino acid aqueous solution. The amino acid aqueous solution may be an aqueous glycine solution, for example. During hydration, the amino acid molecules in the solution, being anions, become intercalated between the layers of the hydrotalcite compound, causing detachment between the layers of the hydrotalcite compound. Detachment between the layers of the hydrotalcite compound results in formation of particles to a very small thickness. The particles with small thickness are heated in the heating step, forming a complex of a hydrotalcite compound in which growth of the particles is promoted in the widthwise direction. In other words, particles with a high aspect ratio are formed. If the amount of amino acids with respect to the amount of hydrotalcite compound is too low, a residue of insufficiently detached hydrotalcite compound particles will remain, resulting in a lower aspect ratio for the resulting complex. The complex therefore has an aspect ratio of lower than 85.

As mentioned above, the complex of the embodiment has a Y.I. value of 0 to 5, as the value representing the yellowness index. The Y.I. value is an index of the “polyamino acid content” which affects coloration such as yellowing. If the Y.I. value is higher than 5, yellowish-brown and similar coloration can occur, potentially impairing the transparency and aesthetic quality when the covering layer is formed using the colored complex as a component of the coating solution. A preferred range for the Y.I. value is 4 or lower, and even more preferably 3 or lower. If the Y.I. value is within this range it will be possible to form a more highly transparent covering layer.

The components in the complex of the embodiment will now be explained.

### (Hydrotalcite Compound)

The hydrotalcite compound in the complex of the embodiment is not particularly restricted, and for example, it may be a hydrotalcite compound represented by the following formula (1).

(M²⁺)_{1-X}(M³⁺)_{X}(OH)₂(Aⁿ⁻)_{X/n}·mH₂O (1)

(In formula (1), M²⁺ represents a divalent metal cation, M³⁺ represents a trivalent metal cation, and Aⁿ⁻ represents an n-valent anion. X is a number satisfying the inequality 0.17<X<0.36. n is an integer of 1 to 6, and m is a number satisfying the inequality 0<m<1.80.)

In formula (1), M²⁺ is preferably Mg²⁺, and M³⁺ is preferably Al³⁺. Because these hydrotalcite compounds are highly safe for the human body and have refractive indexes similar to those of resins such as polypropylene or polyethylene, it is easy to maintain transparency when forming covering layers on substrates made of such resins. The molar ratio of Mg/Al₂ is preferably in the range of 4 to 8 from the viewpoint of more reliably obtaining a hydrotalcite structure.

The type of anion for Aⁿ⁻ in formula (1) is not particularly restricted, and it may be carbonate ion (CO₃²⁻) or hydroxide ion (OH⁻), for example.

The aforementioned hydrotalcite compounds are hydrotalcite compounds to be included in the complex of the embodiment, but the same applies for the hydrotalcite compound precursor to be used as starting material for production of the complex of the embodiment. As explained below, the hydrotalcite compound precursor is calcinated to obtain a complex metal oxide, and then mixed with the amino acid aqueous solution. The hydrotalcite compound precursor is preferably carbonate ion from the viewpoint of avoiding generation of corrosive gases such as chlorine gas and nitrogen dioxide gas during calcination of the hydrotalcite compound precursor, i.e. the hydrotalcite compound before calcination.

### (Amino Acids)

The amino acids to be included in the complex of the embodiment are not particularly restricted and may be any types of amino acids such as α-amino acids, β-amino acids or γ-amino acids. More specific examples include aspartic acid, glutamic acid, asparagine, serine, glycine, β-alanine, β-aminobutyric acid, γ-aminobutyric acid and β-leucine. These amino acids may be all a single type of amino acid, or two or more types of amino acids. The amino acids may also be multimers (peptides) comprising multiple amino acids bonded together.

Preferred among the aforementioned amino acid types, as amino acids to be included in the complex of the embodiment, are amino acids with a solubility of 10 g/100 mL H₂O or greater, and especially glycine compounds. Such amino acids, and especially glycine compounds, have high permittivity and are therefore advantageous for obtaining a complex of primary particles with small thickness. Glycine which exhibits bacteriostatic action is used as a supplement, coloring agent and aromatic component, and is advantageous in terms of biostability.

As used herein, the term “amino acid compound” will be used to refer to substances including amino acids and amino acid multimers. For example, a substance including glycine and polyglycine will be referred to as a “glycine compound”.

The phrase “solubility of 10 g/100 mL H₂O or greater” when used herein means solubility such that the mass of substance (amino acid) dissolving in 100 g of water at 25° C. is 10 g or greater.

The amino acid content in the complex of the embodiment is greater than 0 mass % and 10.0 mass % or lower, with respect to the total mass of the complex. From the viewpoint of allowing more reliable formation of a covering layer exhibiting both high gas barrier properties and transparency, the amino acid content is preferably 1.0 mass % to 8.0 mass % and even more preferably 1.5 mass % to 6.0 mass %.

The complex of the embodiment has a high aspect ratio of 85 or higher, as mentioned above. This allows the complex of the embodiment to exhibit excellent gas barrier properties when used to form a covering layer. The aspect ratio of the complex is preferably 90 or higher and even more preferably 100 or higher from the viewpoint of higher gas barrier properties. The upper limit for the aspect ratio of the complex is not particularly restricted but may be 500 or lower, for example.

For the present purpose, the aspect ratio of the complex is the ratio between the primary particle width (diameter) and thickness in the complex with a layered structure, and it can be determined by dividing the width of the primary particles in the complex by the thickness.

If the complex of the embodiment has an aspect ratio of 85 or higher, the primary particle thickness is not particularly restricted but may be a thickness of 20 nm or smaller, for example. After having formed the covering layer, the complex preferably has a complex primary particle thickness of 20 nm or smaller and an aspect ratio of 100 or higher, from the viewpoint of obtaining superior gas barrier properties.

The complex primary particle thickness is more preferably in the range of 0.7 nm to 10 nm. If the complex primary particle thickness is in the range of 0.7 nm or larger and 10 nm or smaller, then the hydrotalcite compound will be sufficiently detached and will be able to exhibit higher gas barrier properties after forming the covering layer.

The method for producing the complex of the embodiment will now be explained.

### [Method for Producing Complex]

As mentioned above, the complex of the embodiment can be obtained by a production method comprising a solution preparation step, in which a slurry solution comprising a mixture of an amino acid aqueous solution with a prescribed concentration as the release agent, and a complex metal oxide obtained by calcinating a hydrotalcite compound precursor, are mixed; and a heating step in which the slurry solution obtained in the solution preparation step is heated.

The amino acid concentration of the slurry solution in the solution preparation step solution preparation step may be a concentration such that the amino acid content is greater than 0 mass % and 50.0 mass % or lower with respect to the total mass of the complex that is obtained.

### (Solution Preparation Step)

In the solution preparation step, first the hydrotalcite compound precursor is calcinated to obtain a complex metal oxide. The hydrotalcite compound precursor used may be the same hydrotalcite compound as the hydrotalcite compound in the complex. The calcination time for calcination of the hydrotalcite compound precursor is not particularly restricted, and for calcination using a calcination furnace it may be a time of 0.1 to 24 hours, for example. The calcination temperature is also not particularly restricted, and for calcination using a calcination furnace it may be a temperature of 300° C. to 700° C., for example. The calcination time for calcination of the hydrotalcite compound precursor may be a time of 1 minute or longer and 12 hours or less for calcination using microwaves, for example. When the calcination uses microwaves, it may be carried out at a temperature of 300° C. to 700° C., for example.

An amino acid aqueous solution at a prescribed concentration is added to and mixed with the complex metal oxide powder obtained by calcination of the hydrotalcite compound precursor, to obtain a slurry solution. The slurry concentration of the slurry solution is preferably lower than 30 g/L. Since the hydrotalcite compound obtained using such a release agent normally has a small primary particle thickness of several nm, a high slurry concentration often results in gelation during the releasing process, producing a non-homogeneous sample. In the method for producing the complex of the embodiment, the slurry concentration during the solution preparation step may be set to lower than 30 g/L to reduce interaction between the particles of the hydrotalcite compound, thus helping to inhibit gelation during the detachment process. The production method can therefore maintain a slurry state for the slurry solution, so that a complex can be homogeneously and effectively produced.

The slurry concentration during the solution preparation step is more preferably in the range of 10 g/L to 28 g/L from the viewpoint of productivity. The slurry concentration can be determined by the following formula.

$$Slurry concentration ( g / L ) = Complex metal oxide weight ( g ) / slurry voume ( L )$$

The amino acid concentration of the slurry solution in the solution preparation step is preferably lower than 0.6 mol/L. If the amino acid concentration of the slurry solution is within this range it will be possible to more reliably obtain a complex having an aspect ratio of 85 or higher and a Y.I. value of 0 to 5. In other words, it will be possible to more reliably obtain a complex that can form a highly transparent covering layer having a high gas barrier property and no coloration such as yellowish-brown coloration. The amino acid concentration of the slurry solution is more preferably in the range of 0.2 mol/L to 0.5 mol/L.

In the solution preparation step, the molar ratio between the amino acids in the slurry solution and the trivalent metal cation of the hydrotalcite compound (the molar ratio of amino acid/(M³⁺)₂) is preferably in the range of 2 to 6. For example, when the amino acids are glycine and the trivalent metal cation of the hydrotalcite compound is Al³⁺ the molar ratio of glycine/Al₂ is preferably in the range of 2 to 6. If the molar ratio is within this range it will be possible to more reliably obtain a complex with an aspect ratio of 85 or higher and a Y.I. value of 0 to 5, i.e. a complex that can form a highly transparent covering layer having a high gas barrier property and no coloration such as yellowish-brown coloration.

### (Heating Step)

In the heating step, the slurry solution obtained in the solution preparation step is heated to promote particle growth of the hydrotalcite compound. The amino acid molecules in the solution are incorporated as anions between the hydrotalcite compound layers, causing the hydrotalcite compound layers to detach and forming a complex of the hydrotalcite compound and amino acids as thin particles (that is, particles with a high aspect ratio).

Heating in the heating step is preferably carried out while stirring the slurry solution. The stirring means is not particularly restricted, but preferably it allows uniform stirring to be accomplished while maintaining the fluidity of the slurry solution.

The heating temperature in the heating step is not particularly restricted but is preferably a temperature in the range of 20° C. to 250° C., more preferably the upper limit of the heating temperature being 170° C. from the viewpoint of allowing denaturation of the amino acids to be more reliably inhibited. The heating time is not particularly restricted, but it is preferably a time in the range of 1 minutes to 100 hours. The heating step is preferably carried out in a closed system from the viewpoint of helping to prevent variation in the concentration of the slurry solution.

### (Other Steps)

The steps following the heating step are not particularly restricted so long as the effect of the invention is not hindered. Examples of steps after the heating step include a washing step in which the slurry solution is washed with an alkali solution (such as a sodium hydroxide solution) after the heating step, a solid-liquid separation step in which solid-liquid separation treatment is carried out after the washing step to obtain a solid, a drying step in which the solid obtained by the solid-liquid separation step is dried to obtain a powder of the complex, and a surface treatment step in which the surfaces of the complex particles are treated with any of various surface treatment agents.

The surface treatment agent used for the surface treatment step is not particularly restricted, and examples include anionic surfactants, cationic surfactants, phosphoric acid ester treatment agents, silane coupling agents, titanate coupling agents, aluminum coupling agents, silicone-based treatment agents, silicic acid and water glass. Particularly preferred surface treatment agents are one or more selected from the group consisting of oleic acid, stearic acid, octanoic acid and octylic acid. Surface treatment of the complex particles can prevent aggregation of the primary particles during addition, kneading and dispersion in resins.

A dilution step may also be carried out before the washing step for dilution of the heated slurry with ion-exchanged water, or a coating solution preparation step may be carried out instead of the drying step, for mixing with the polymer solution to prepare a coating solution.

The complex of the embodiment obtained by the production method described above may be mixed with a polymer solution as explained above, to prepare a coating solution.

### [Coating Solution]

The coating solution comprising the complex of the embodiment and a polymer can be coated and dried on a substrate as described below, to form a covering layer. The concentration of the complex in the coating solution is not particularly restricted, but from the viewpoint of coatability onto substrates it is preferably a concentration of 20 mass % or lower and more preferably 10 mass % or lower.

### (Polymer)

The polymer in the coating solution may be used as a solution dissolved in a solvent such as water or an alcohol. The polymer concentration in the solution is not particularly restricted, but from the viewpoint of coatability onto substrates it is preferably a concentration of 20 mass % or lower and more preferably 10 mass % or lower.

The type of polymer in the coating solution is not particularly restricted, and may be a polymer suited for the purpose of the covering layer or film to be formed. Examples of such polymers include water-soluble polymers, and more specifically polyvinyl alcohol, vinyl alcohol-containing copolymers (such as polyethylene vinyl alcohol), carboxymethyl cellulose, polyacrylic acid and polyacrylamide. These water-soluble polymers may be used alone as single types of polymers, or as combinations of two or more types of polymers. Using a water-soluble polymer can facilitate separation between the substrate and the coating layer while increasing the recycling property, thus helping to contribute to achieving SDGs (Sustainable Development Goals) adopted at United Nations summits.

Polyvinyl alcohol is preferably used among the aforementioned water-soluble polymers from the viewpoint of the gas barrier property, transparency and coatability.

### (Other Components)

The coating solution may also comprise other added components in addition to the complex and polymer, within ranges that do not interfere with the effect of the invention. Such added components are not particularly restricted, and examples include antioxidants, reinforcing agents, ultraviolet absorbers, pigments, crosslinking agents and flame retardants. Incidentally, any of these added components may be used as single types alone, or two or more different added components may be used in combination.

### [Film]

The coating solution comprising the complex of the embodiment and a polymer can be coated and dried on a substrate to form a covering layer. The drying conditions may be appropriately set in a temperature range from ordinary temperature to 160° C. for a period from 1 second to 24 hours, for example. If a film-like substrate (hereunder also referred to as “film substrate”) is used as the substrate, it will be possible to obtain a film having a multilayer structure where the film substrate is covered by the covering layer. The covering layer on the substrate may also be released to obtain a film with a single layer structure comprising the covering layer. A film with a multilayer structure or single layer structure obtained in this manner can be suitably used as an arbitrary type of packaging film, for example.

When using a water-soluble polymer, the packaging film may be formed as a multilayer structure of the water-soluble polymer or a single layer structure of the water-soluble polymer. Examples of such packaging films include films used for packaging of chemical agents such as detergents, agricultural chemicals or drugs. Examples of drug forms include powders, solids, gels and liquids. A packaging film may also be used as a film for packaging of fish bait. A packaging film may also be used as a film for packaging of liquid detergent.

### (Substrate)

The substrate to be used to form a film is not particularly restricted, and any substrate may be used depending on the purpose of use of the formed film. Examples of such substrates include the film substrates mentioned above, and more specifically, they include resin-based films including polyolefin films such as polyethylene or polypropylene and polyester films such as polyethylene terephthalate; paper; and fiber sheets such as woven fabrics or nonwoven fabrics and knitted fabrics.

When a film substrate is used as the substrate, the thickness is not particularly restricted but is preferably 0.01 μm to 250 μm, and more preferably in the range of 1 μm to 100 μm, for example. The thickness of the covering layer formed by the coating solution is also not particularly restricted and may be a thickness in the range of 0.01 μm to 100 μm, for example.

The film to be obtained using the complex of the invention can be used in a wide range of fields including as a food packaging film, beverage packaging film, beverage bottle, drug packaging film, industrial gas barrier film, gas separation film or paper barrier material.

The film containing the complex of the invention may be used as a bag-shaped packaging container. The packaging container may have any desired construction suited for an individual packaging or an interior or exterior finishing, as well as for the items to be packaged inside.

When the film containing the complex of the invention is to be used as a bag-shaped packaging container, the bag-shaped packaging container may have a construction with a covering layer on the outer side of the packaging container, for example. The covering layer can be formed by coating and drying the complex-containing coating solution on the outer side of the packaging container. The packaging container may have a construction with a high aspect ratio, and thus an increased gas barrier property, as well as reduced colorability. In particular, the packaging container may have a covering layer formed by the coating solution on the outer side of the packaging container, thereby preventing elution of the coating components even if the packaged contents are liquid or contain liquid.

When the film containing the complex of the invention is to be used as a bag-shaped packaging container, the bag-shaped package may also have a construction with a covering layer on the inner side of the packaging container, for example. The covering layer can be formed by coating and drying the complex-containing coating solution on the inner side of the packaging container. The packaging container may have the covering layer formed by the coating solution on the inner side of the packaging container so that the covering layer is maintained without damage even when the packaging container has been subjected to external effects such as friction or wetting with water. As a result, the packaging container maintains the effects of the covering layer such as its gas barrier property, allowing the quality of the contents to be preserved. When the bag-shaped package has a construction with the covering layer on the inner side of the packaging container, the covering layer may be formed on the non-fused sections of the packaging container. Moreover, when the bag-shaped package has a construction with the covering layer on the inner side of the packaging container, an adhesive layer with a gas barrier property may be formed on the fused surface sections of the packaging container, with the covering layer being formed on the non-adhesive layer-formed sections.

Alternatively, when the film containing the complex of the invention is to be used as a bag-shaped packaging container, the bag-shaped package may have a construction with a covering layer on both the inner side and outer side of the packaging container, for example. If the bag-shaped package has a covering layer formed by the coating solution on both the inner side and outer side of the packaging container, it will be possible to obtain higher gas barrier properties compared to when the covering layer is only on either the inner side or the outer side. As a result, the bag-shaped packaging container can help maintain quality and prolong viable storage of low-moisture-active contents such as wheat flour, confectioneries, tea leaves and dried vegetables.

The film containing the complex of the invention may also be formed as a multilayer film structure having a covering layer formed by the coating solution between film substrates. When a multilayer film containing the complex of the invention is used as a bag-shaped packaging container, the bag-shaped package may have a construction in which the packaged contents are housed internally within a pair of multilayer films, for example. In the bag-shaped packaging container which uses a multilayer film containing the complex, the film substrates can be bonded together, thus providing a construction that does not require separate use of an adhesive. A bag-shaped packaging container that uses a multilayer film can exhibit the advantages of both a bag-shaped packaging container having a covering layer formed by a coating solution on the outer side of the packaging container, and a bag-shaped packaging container having a covering layer formed by a coating solution on the inner side of the packaging container.

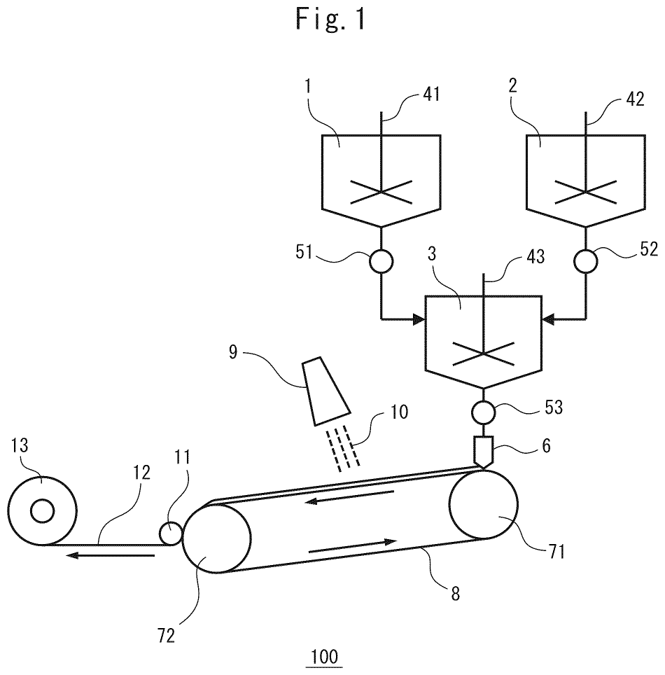

When the complex-containing film has a single layer structure, the covering layer itself serves as the film. A single layer structure film can be produced using the film production apparatus 100 shown in FIG. 1, for example.

The film production apparatus 100 comprises a first tank 1, a second tank 2 and a third tank 3. The film production apparatus 100 also comprises a first coating drum 71, a second coating drum 72 and a casting belt 8. The film production apparatus 100 rotates the first coating drum 71 and second coating drum 72, thereby moving the casting belt 8 between the first coating drum 71 and second coating drum 72. In other words, the casting belt 8 is constructed so that rotation of the first coating drum 71 and second coating drum 72 allows it to move between the first coating drum 71 and second coating drum 72. The film production apparatus 100 is provided with a drying apparatus 9 that can blow hot air 10 onto the casting belt 8 that moves between the first coating drum 71 and second coating drum 72. FIG. 1 shows the hot air 10 as a dashed line.

The first tank 1 of FIG. 1 stores a slurry containing the complex and water. A first stirrer 41 that stirs the slurry is provided in the first tank 1. The second tank 2 stores a polymer. A second stirrer 42 that stirs the polymer is provided in the second tank 2. The first tank 1 is constructed so as to supply the slurry to the third tank 3 via a first pump 51. The second tank 2 is constructed so as to supply the polymer to the third tank 3 via a second pump 52. The third tank 3 is provided with a third stirrer 43 that stirs a polymer blend obtained by mixing the slurry supplied from the first tank 1 and the polymer supplied from the second tank 2.

The third tank 3 casts the polymer blend onto the casting belt 8 from a casting die 6, through the third pump 53. The polymer blend cast from the casting die 6 moves with the casting belt 8 in the traveling direction, while disposed in the widthwise direction and traveling direction of the casting belt 8. The polymer blend which moves in the traveling direction together with the casting belt 8 is dried by the hot air 10 from the drying apparatus 9, forming the film 12. The arrow shown parallel to the casting belt 8 in FIG. 1 indicates the traveling direction of the casting belt 8.

The film production apparatus 100 has, adjacent to the second coating drum 72, a release roll 11 which releases the film 12 on the casting belt 8. The film production apparatus 100 has a winding apparatus 13 provided for the release roll 11. The winding apparatus 13 winds up the film 12 that has been released at the release roll 11. The arrow shown parallel to the film 12 released at the release roll 11 in FIG. 1 indicates the traveling direction of the film 12.

The film production apparatus 100 may also employ a plated roll as the support instead of the casting belt 8. The film is not limited to having a construction obtained by solution casting, and may also be formed by melt extrusion.

A packaging film using a covering layer containing a complex of the embodiment may have both ends of the film sealed together using a heated seal bar, for example, to form a bag-shaped packaging container internally enclosing the contents. The film sealing method may be heat sealing, water sealing or glue sealing, for example.

The packaging film may seal and package a single portion of liquid detergent, for example. When a polyvinyl alcohol is used, the covering layer is a water-soluble bag-shaped package. The bag-shaped package has the water solubility of the polyvinyl alcohol while also maintaining high aroma retention since it comprises the complex of the embodiment. By comprising the complex, the bag-shaped package can be formed as a thin-film bag-shaped package while retaining strength. With a water-soluble bag-shaped packaging container, the film thickness is preferably 1 μm or larger and more preferably 10 μm or larger. With a water-soluble bag-shaped packaging container, the film thickness is preferably 1000 μm or smaller and more preferably 500 μm or smaller. The film thickness can be measured using a micrometer or a film thickness meter based on optical interferometry.

The coating solution may also comprise one or more different auxiliary agents such as a surfactant, plasticizer, release agent, stabilizer, coloring agent, ultraviolet absorber, extender, antifoaming agent or dispersing agent, for example. The auxiliary agent may be an organic substance or an inorganic substance.

The surfactant may be an ionic surfactant, nonionic surfactant or polymer surfactant, for example. An ionic surfactant may be an anionic surfactant, cationic surfactant or amphoteric surfactant, for example. The surfactant content is not particularly restricted but is preferably 0.03 parts by mass or greater and more preferably 0.05 parts by mass or greater, for example, with respect to 100 parts by mass of the polymer. The surfactant content is also preferably 2.5 parts by mass or lower and more preferably 1.5 parts by mass or lower, for example, with respect to 100 parts by mass of the polymer.

Examples of plasticizers include glycerin, diglycerin, glucose, fructose, lactose, sorbitol, mannitol, ethylene glycol, propylene glycol and trimethylolpropane.

The coating solution may also include inorganic particles in addition to the complex comprising the hydrotalcite compound and amino acids. Examples of inorganic particles in addition to the complex include silica, talc, kaolin, mica, graphite, calcium sulfate, magnesium oxide and magnesium hydroxide.

### [Examples of Non-Film Applications]

The complex of the invention can also be utilized as an additive for addition to cosmetic products. In other words, one aspect of the invention is a cosmetic product comprising a complex of the invention. The complex controls permeation of characteristic molecules such as oxygen and water. By adjusting the complex content in a cosmetic product containing the complex it is possible to control the permeability of characteristic molecules such as oxygen and water. The complex exhibits a specific yellowness index, and reflects light. With a cosmetic product comprising the complex, therefore, it is possible to produce a cosmetic product that is able to control light reflecting properties.

The complex of the invention can also be utilized as an additive for addition to coating materials. In other words, one aspect of the invention is a coating material comprising a complex of the invention. The complex inhibits permeation of characteristic molecules such as oxygen and water vapor. By adjusting the complex content in a coating material containing the complex it is possible to control the permeability of characteristic molecules such as oxygen and water vapor. The complex has a flame-retardant property due to the hydrotalcite compound. With a coating material comprising the complex, therefore, it is possible to produce a coating material that is able to reduce combustion. The complex also exhibits a specific yellowness index, and reflects light. With a coating material comprising the complex, therefore, it is possible to produce a coating material that is able to control light reflecting properties.

The complex of the invention can also be utilized as a resin additive for addition to resins. In other words, one aspect of the invention is a resin composition comprising a complex of the invention. The resin composition comprising the complex as a resin additive can exhibit higher strength than a resin composition without the complex. In other words, using the complex as a resin additive can yield a resin composition having a reinforcing effect due to a high aspect ratio. A resin composition comprising the complex as a resin additive can also exhibit coloration to a predetermined color matching the yellowness index of the complex, by adjusting the content of the complex.

The resin composition comprising the complex of the invention can be molded into shapes suitable for appropriate uses. It can be molded into a tube shape, for example, by extrusion molding. In other words, one aspect of the invention is a tube comprising a complex of the invention. The complex inhibits permeation of characteristic molecules including inert gases such as helium gas, or oxygen and water. By adjusting the complex content in a tube containing the complex it is possible to control the permeability of characteristic molecules including inert gases such as helium gas, or oxygen and water. A tube obtained by combining a complex of the invention with a highly biocompatible resin, for example, can be used as a tube requiring gas barrier properties for use in the field of medicine. An example of such a tube is a medical catheter.

The invention is not restricted to the embodiments described above or the Examples described below, and can incorporate appropriate combinations, substitutions and modifications within ranges that do not fall outside of the object and gist of the invention.

### EXAMPLES

The invention will now be explained in greater detail using Examples and Comparative Examples, with the understanding that the invention is not limited only to these Examples.

### Example 1

### (Production of Hydrotalcite Compound Precursor)

After placing ion-exchanged water in a 1 L volume reaction tank, 160 mL of a 1.5 mol/L magnesium chloride aqueous solution, 120 mL of a 1 mol/L aluminum chloride aqueous solution and a liquid mixture of 90 mL of an 8 mol/L sodium hydroxide aqueous solution with 60 mL of a 1 mol/L sodium carbonate aqueous solution were simultaneously added dropwise while stirring, to obtain a reaction product. The pH during the reaction was 9.5. After washing the obtained reaction product, ion-exchanged water was added to obtain 700 mL of a re-emulsified slurry. The re-emulsified slurry was subjected to hydrothermal treatment for 13 hours at 170° C. The obtained solid was washed with water and then dried for 16 hours at 105° C. and pulverized. The obtained powder was a hydrotalcite compound represented by the chemical formula Mg_{0.67}Al_{0.33}(OH)₂(CO₃)_{0.17}·0.50H₂O. The hydrotalcite compound was used as a hydrotalcite compound precursor for production of a complex.

### (Calcination of Hydrotalcite Compound Precursor)

The obtained hydrotalcite compound precursor was calcinated in an electric furnace for 12 hours at 450° C. to obtain a calcinated product (complex metal oxide).

### (Solution Preparation Step)

A 17.5 g portion of the obtained calcinated product was loaded into a glass beaker, 175 mL of a 2 mol/L glycine aqueous solution was added (corresponding to 26.3 g of glycine powder), and the mixture was stirred to uniformity. The glycine/Al₂ molar ratio of the obtained slurry solution was 5.26. After then adding ion-exchanged water to a total volume of 700 mL, stirring was further carried out to uniformity. The slurry concentration was 25 g/L. The glycine concentration was 0.5 mol/L.

### (Heating Step)

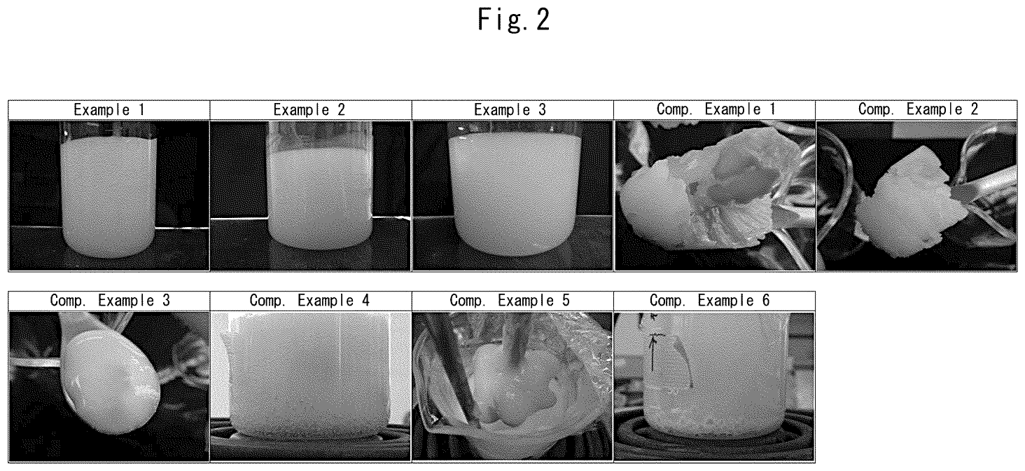

The slurry solution was then stirred at 700 rpm while conducting hydrothermal treatment for 48 hours at 100° C. The obtained sample (slurry solution) was a white slurry with no particular noticeable odor. FIG. 2 shows a photograph of a slurry solution after the heating step.

### (Washing Step to Drying Step)

After adding ion-exchanged water to the obtained slurry solution to a total volume of 1750 mL, a stirrer was used for 16 hours of stirring at 400 rpm, ordinary temperature. While continuing to stir the slurry solution, 88.4 mL of a 3.96 mol/L sodium hydroxide aqueous solution was slowly added dropwise to the slurry solution. The resulting slurry solution was treated for solid-liquid separation to obtain a solid. The obtained solid was dried at 60° C. for 16 hours to obtain a sample (complex) for Example 1. The features of the obtained sample are shown in Table 1.

### Example 2

A sample for Example 2 was obtained in the same manner as Example 1, except that in the solution preparation step, 14.0 g of calcinated product and 140 mL of a 2 mol/L glycine aqueous solution (corresponding to 21.0 g of glycine powder) were used. The slurry concentration of the slurry solution in the solution preparation step was 20 g/L, and the glycine concentration was 0.4 mol/L.

### Example 3

A sample for Example 3 was obtained in the same manner as Example 1, except that in the solution preparation step, 7.0 g of calcinated product and 70 mL of a 2 mol/L glycine aqueous solution (corresponding to 10.5 g of glycine powder) were used. The slurry concentration of the slurry solution in the solution preparation step was 10 g/L, and the glycine concentration was 0.2 mol/L.

### Comparative Example 1

A sample for Comparative Example 1 was obtained in the same manner as Example 1, except that in the solution preparation step, 70.0 g of calcinated product and 700 mL of a 2 mol/L glycine aqueous solution (corresponding to 105.1 g of glycine powder) were used, and stirring was not carried out during the heating step. The slurry concentration of the slurry solution in the solution preparation step was 100 g/L, and the glycine concentration was 2 mol/L.

### Comparative Example 2

A sample for Comparative Example 2 was obtained in the same manner as Example 1, except that in the solution preparation step, 70.0 g of calcinated product and 266 mL of a 2 mol/L glycine aqueous solution (corresponding to 39.9 g of glycine powder) were used, and stirring was not carried out during the heating step. The slurry concentration of the slurry solution in the solution preparation step was 100 g/L, and the glycine concentration was 0.76 mol/L.

### Comparative Example 3

A sample for Comparative Example 3 was obtained in the same manner as Example 1, except that in the solution preparation step, 70.0 g of calcinated product was used, no glycine aqueous solution was used, stirring was not carried out during the heating step, and no washing step was carried out. The slurry concentration of the slurry solution in the solution preparation step was 100 g/L, and the glycine concentration was 0 mol/L.

### Comparative Example 4

A sample for Comparative Example 4 was obtained in the same manner as Example 1, except that in the solution preparation step, 35.0 g of calcinated product and 350 mL of a 2 mol/L glycine aqueous solution (corresponding to 52.5 g of glycine powder) were used. The slurry concentration of the slurry solution in the solution preparation step was 50 g/L, and the glycine concentration was 1 mol/L.

### Comparative Example 5

A sample for Comparative Example 5 was obtained in the same manner as Example 1, except that in the solution preparation step, 28.0 g of calcinated product and 280 mL of a 2 mol/L glycine aqueous solution (corresponding to 42.0 g of glycine powder) were used. The slurry concentration of the slurry solution in the solution preparation step was 40 g/L, and the glycine concentration was 0.8 mol/L.

### Comparative Example 6

A sample for Comparative Example 6 was obtained in the same manner as Example 1, except that in the solution preparation step, 21.0 g of calcinated product and 210 mL of a 2 mol/L glycine aqueous solution (corresponding to 31.5 g of glycine powder) were used. The slurry concentration of the slurry solution in the solution preparation step was 30 g/L, and the glycine concentration was 0.6 mol/L.

### Example 4

### (Preparation of Coating Solution)

The sample of Example 1 and a polyvinyl alcohol (PVA) aqueous solution (Mowiol® 8-88, Mw: 67,000, product of Sigma-Aldrich Japan, KK.) were measured out in solid concentrations, adding ion-exchanged water for preparation to 3 wt % of the complex of the hydrotalcite compound and glycine compound of Example 1 and 2 wt % of PVA, and the mixture was stirred to obtain a coating solution.

### (Fabrication of Film)

First, a polyethylene terephthalate (PET) film (LUMIRROR® by Toray Co., Ltd., film thickness: 50 μm, product number type: #50-U483) cut out to 55 mm length×105 mm width, as a film substrate, was anchored to a glass plate of an automatic coating apparatus (Model PI-1210 by Tester Sangyo Co., Ltd.). A baker applicator (Model YBA by Yoshimitsu Seiki Co.) was then set on the glass plate of the automatic coating apparatus, and the coating solution was coated onto a PET film to a uniform thickness of 50 μm. The covering layer formed by the coated solution was dried for 16 hours at ordinary temperature to obtain a film for Example 4.

### Comparative Example 7

A film for Comparative Example 7 was obtained in the same manner as Example 4, except that the sample of Comparative Example 1 was used instead of the sample of Example 1.

### Comparative Example 8

A film for Comparative Example 8 was obtained in the same manner as Example 4, except that the hydrotalcite compound precursor obtained in the step of “Production of hydrotalcite compound precursor” in Example 1 was used instead of the sample of Example 1 (complex of a hydrotalcite compound and glycine compound). In other words, the film of Comparative Example 8 used an unseparated hydrotalcite compound (unseparated HT) instead of a complex of a hydrotalcite compound and glycine compound.

### Comparative Example 9

A film for Comparative Example 9 was obtained in the same manner as Example 4, except that no coating solution was applied. In other words, the film of Comparative Example 9 was a simple PET film (film substrate) without formation of a covering layer of a coating solution.

### Comparative Example 10

A film for Comparative Example 10 was obtained in the same manner as Example 4, except that a coating solution comprising PVA alone was used.

### Example 5

### (Fabrication of Film)

A film for Example 5 was obtained in the same manner as Example 4, except that a polypropylene (PP) film (PYLEN® Film-OT by Toyobo, Ltd., film thickness: 50 μm, grade: P2261) was used as the film substrate, and the coating solution was applied to a uniform thickness of 25 μm.

### Example 6

A film for Example 6 was obtained in the same manner as Example 5, except that the coating solution was applied to a uniform thickness of 12.5 μm.

### Comparative Example 11

A film for Comparative Example 11 was obtained in the same manner as Example 5, except that no coating solution was applied. In other words, the film of Comparative Example 11 was a simple PP film (film substrate) without formation of a covering layer of a coating solution.

### Comparative Example 12

A film for Comparative Example 12 was obtained in the same manner as Example 5, except that a coating solution comprising PVA alone was used.

### [Measurements]

The following physical properties of the samples from Example 1 to Example 3 and from Comparative Example 1 to Comparative Example 6 were measured. The measurement results are shown in Table 1 below. Photographs were taken of slurry solutions after the heating step, for Example 1 to Example 3 and Comparative Example 1 to Comparative Example 3, and slurry solutions after heating to 100° C. in the heating step, for Comparative Example 4 to Comparative Example 6. The photographs are shown in FIG. 2.

(Viscosity Measurement after Heating Step)

After adjusting the slurry solution to a temperature of 25° C., a Brookfield viscometer (Model DV2T by Brookfield) was used to measure the viscosity at the same temperature.

### (Measurement of Primary Particle Thickness and Aspect Ratio of Complex)

A scanning probe microscope (Model SPM-9700HT by Shimadzu Corp.) was used to measure the primary particle width and thickness. The aspect ratio was calculated by the following formula.

$$Primary particle aspect ratio = ( Primary particle width ) / ( primary particle thickness )$$

The aspect ratio was determined by calculating the aspect ratio for each of 20 arbitrary primary particles, and taking the average as the aspect ratio of the sample.

### (Measurement of Glycine Compound Content)

After mixing 0.5 g of a powder sample dried at 60° C. for 16 hours, 5 g of potassium sulfate and 2 g of copper (II) sulfate pentahydrate and placing the mixture in a sample tube, 15 ml of concentrated sulfuric acid was added. A Kjeldahl Analysis System (KjelFlex K-360/K-425 SpeedDigester by Buchi Co.) was then used for 90 minutes of treatment at 470° C. Next, 10 mL of ion-exchanged water, a 30% sodium hydroxide aqueous solution and 40 mL of a 2% boric acid aqueous solution were used for steam distillation for 240 seconds. Three drops of a mixed indicator of methyl red and methylene blue were added to the obtained distillate, and a 0.1 mol/L hydrochloric acid aqueous solution was used for titration until the distillate turned purple. A blank without the powder sample was also used for titration in the same manner, and the glycine compound content was calculated by the following formulas (I) and (II).

$$Nitrogen N content [ wt \% ] = ( 0.1 mol / L hydrochloric acid solution factor ) \times ( drip volume [ ml ] - blank [ ml ] ) \times ( 1.401 [ mg / ml ] ) \times 100 / powder sample mass [ mg ] \qquad ( I )$$

$$Glycine compound content [ wt \% ] = Nitrogen content [ wt \% ] \times ( 75.07 [ g / mol ] / 14.01 [ g / mol ] ) \qquad ( II )$$

### (Measurement of Yellowness Index (Y.I. Value))

The powder sample that had been dried at 60° C. for 16 hours was pulverized and screened through a screen net with a 150 μm mesh, after which 0.2 g was placed in a glass reagent bottle. Next, a 100 viewport and sample stage were used for measurement with a color measurement color meter (Model ZE6000 Color meter by Nippon Denshoku Industries Co., Ltd.) which had been previously calibrated using a standard white board. Measurement was conducted a total of 3 times, shaking the reagent bottle 10 times before each measurement. The average value was calculated for three measurements to obtain the Yellow Index value (Y.I. value).

### [Film Evaluation]

The following physical properties of the films of Example 4 to Example 6 and Comparative Example 7 to Comparative Example 12 were measured. The measurement results are shown in Table 2 and Table 3 below.

### (Measurement of Total Light Transmittance and Haze (Cloudiness))

The film was cut at 20 mm from the coating end point, and a cloudiness meter (Model NDH4000 by Nippon Denshoku Industries Co., Ltd.) was used for measurement with the coated side as the light-incident side, to obtain the total light transmittance and haze (cloudiness).

### (Measurement of Oxygen Gas Permeability)

The film was punched out to a circular shape with a diameter of about 55 mm, and a gas permeability tester (Model GTR-11AET by GTR Tec Corp.) was used to measure the gas permeability. The measuring method was carried out under the following conditions, according to JIS K 7126-1, Part 1 (“Differential pressure method”).
Measuring temperature: 23.0° C.
Relative humidity: zero
Permeation area: 15.2 cm²
Measuring gas: oxygen gas

The oxygen gas permeating through the film was analyzed using a capillary gas chromatography system (Model GC-2014 by Shimadzu Corp.), to obtain the oxygen gas permeability. For oxygen gas permeability measurement, the analysis time can be appropriately adjusted according to the gas permeability of the film.

TABLE 1

| | | | | | Comp. | Comp. |
|---|---|---|---|---|---|---|
| | | Example 1 | Example 2 | Example 3 | Example 1 | Example 2 |
| Complex | Glycine concentration | 0.50 | 0.40 | 0.20 | 2.00 | 0.76 |
| production | (mol/L) | | | | | |
| conditions | Glycine/Al₂ molar ratio | 5.26 | 5.26 | 5.26 | 5.26 | 2.00 |
| | Slurry concentration (g/L) | 25 | 20 | 10 | 100 | 100 |
| | Uniformly stirrable or not | Yes | Yes | Yes | No | No |
| State after | Outer appearance | White | White | White | Surface layer | White |
| heating step | | | | | yellowish- | |
| | | | | | brown, rest | |
| | | | | | white | |
| | Viscosity state | Slurry | Slurry | Slurry | Gel | Gel |
| | Viscosity (cP) | 1820 | 504 | 14.6 | Unmeasurable | Unmeasurable |
| | Odor | None | None | None | Burnt odor | None |
| Complex | Primary particle thickness | 2 | 3 | 3 | 3 | 11 |
| characteristics | (nm) | | | | | |
| | Aspect ratio | 171 | 112 | 114 | 223 | 25 |
| | Glycine compound content | 4.66 | 5.14 | 4.07 | 5.20 | 4.45 |
| | (wt %) | | | | | |
| | Y.I. value | 1.7 | 2.3 | 1.0 | 5.6 | 3.3 |

| | | Comp. | Comp. | Comp. | Comp. |
|---|---|---|---|---|---|
| | | Example 3 | Example 4 | Example 5 | Example 6 |
| Complex | Glycine concentration | 0.00 | 1.00 | 0.80 | 0.60 |
| production | (mol/L) | | | | |
| conditions | Glycine/Al₂ molar ratio | 0.00 | 5.26 | 5.26 | 5.26 |
| | Slurry concentration (g/L) | 100 | 50 | 40 | 30 |
| | Uniformly stirrable or not | Yes | No | No | No |
| State after | Outer appearance | White | Bottom section | Bottom section | Bottom section |
| heating step | | | yellowish- | yellowish- | yellowish- |
| | | | brown, rest | brown, rest | brown, rest |
| | | | white | white | white |
| | Viscosity state | Slurry | Gel | Gel | Gel |
| | Viscosity (cP) | 1.2 | Unmeasurable | Unmeasurable | Unmeasurable |
| | Odor | None | — | — | — |
| Complex | Primary particle thickness | 37 | — | — | — |
| characteristics | (nm) | | | | |
| | Aspect ratio | 6 | — | — | — |
| | Glycine compound content | 0.00 | — | — | — |
| | (wt %) | | | | |
| | Y.I. value | 0.9 | — | — | — |

TABLE 2

| | | | Comp. | Comp. | Comp. | Comp. |
|---|---|---|---|---|---|---|
| | | Example 4 | Example 7 | Example 8 | Example 9 | Example 10 |
| Film fabrication | Added complex | Example 1 | Comp. | Unreleased | None | None |
| conditions | | | Example 1 | HT | | |
| | Coating thickness (μm) | 50.0 | 50.0 | 50.0 | None | 50.0 |
| Film | Total light transmittance | 91.7 | 91.6 | 70.6 | 92.1 | 91.6 |
| characteristics | (%) | | | | | |
| | Haze (cloudiness X %) | 3.5 | 5.1 | 98.8 | 1.1 | 0.9 |

TABLE 3

| | | | | Comp. | Comp. |
|---|---|---|---|---|---|
| | | Example 5 | Example 6 | Example 11 | Example 12 |
| Film fabrication | Added complex | Example 1 | Example 1 | None | None |
| conditions | Coating thickness (μm) | 25.0 | 12.5 | None | 25.0 |
| Film characteristics | O₂ permeability | 0.5 | 0.3 | 802 | 39.6 |
| | [cm³/(m² · day · atm)] | | | | |

As shown in Table 1 to Table 3 and FIG. 2, the complexes of hydrotalcite compounds and glycine compounds according to the Examples of the invention were able to form films (covering layers) exhibiting both high gas barrier properties and transparency. The complexes of the Comparative Examples, on the other hand, were colored yellowish-brown and gelled, and it was not possible to form films exhibiting both high gas barrier properties and transparency as according to the invention.

### INDUSTRIAL APPLICABILITY

A film comprising a complex of the invention, a coating solution comprising the complex, and a covering layer formed using the coating solution, can be utilized as a food packaging film, a beverage packaging film, a beverage bottle, a pharmaceutical packaging film, an industrial gas barrier film or a gas separation film, for example. The complex of the invention and the coating solution comprising the complex can also be suitably used in a wide range of fields including paper barrier materials, coating materials, scratch resistant materials, cosmetic products and additives.

### REFERENCE SIGNS LIST

100 Film production apparatus
1 First tank
2 Second tank
3 Third tank
41 First stirrer
42 Second stirrer
43 Third stirrer
51 First pump
52 Second pump
53 Third pump
6 Casting die
71 First coating drum
72 Second coating drum
8 Casting belt
9 Drying apparatus
10 Hot air
11 Release roll
12 Film
13 Winding apparatus

1. A complex comprising a hydrotalcite compound and an amino acid, having an aspect ratio of 85 or higher, wherein:
a Y.I. value representing a yellowness index of the complex is 0 to 5, and
an amino acid content is greater than 0 mass % and 10.0 mass % or lower with respect to a total mass of the complex.

2. The complex according to claim 1, wherein the amino acid is a glycine compound.

3. The complex according to claim 1, wherein the hydrotalcite compound is represented by formula (1),
(M²⁺)_{1-X}(M³⁺)_{X}(OH)₂(Aⁿ⁻)_{X/n}·mH₂O (1)
(In formula (1), M²⁺ represents a divalent metal cation, M³⁺ represents a trivalent metal cation, Aⁿ⁻ represents an n-valent anion, X represents a number satisfying 0.17<X<0.36, n is an integer of 1 to 6, and m represents a number satisfying 0<m<1.80).

4. The complex according to claim 1, wherein the complex has a primary particle thickness of 20 nm or smaller, and the aspect ratio of 100 or higher.

5. A coating solution comprising the complex according to claim 1 and a polymer.

6. The coating solution according to claim 5, wherein the polymer is a water-soluble polymer.

7. The coating solution according to claim 6, wherein the water-soluble polymer is polyvinyl alcohol.

8. A film comprising a covering layer formed by the coating solution according to claim 5.

9. The film according to claim 8, wherein a covering layer thickness is 1 μm to 1000 μm.
